(19) United States
(12) Patent Application Publication
(10) Pub. No.: US 2015/0209576 A1
(43) Pub. Date: Jul. 30, 2015

(54) DURABLE SMALL GAUGE WIRE ELECTRICAL CONDUCTOR SUITABLE FOR DELIVERY OF HIGH INTENSITY ENERGY PULSES

(71) Applicants: Robert G. Walsh, Newport, OR (US); Jin Shimada, Grantsburg, WI (US); Scott Engle, Independence, MN (US)

(72) Inventors: Robert G. Walsh, Newport, OR (US); Jin Shimada, Grantsburg, WI (US); Scott Engle, Independence, MN (US)

(21) Appl. No.: 14/165,559

(22) Filed: Jan. 27, 2014

(51) Int. Cl. A61N 1/05

(52) U.S. Cl. CPC A61N 1/057; A61N 1/0563

(57) ABSTRACT

Implantable medical devices intended for electrostimulation and sensing devices typically incorporate one or more electrical conductors as leads for electrical stimulation to, or retrieval of localized sensing data from, discrete points in the body, such as the heart. Certain applications require delivery of high intensity electrical pulses, i.e. CRTs, or defibrillators. As described herein a CRT delivers high energy pulses via a durable fine wire lead formed of a glass, silica, sapphire or crystalline quartz fiber core with a metal coating. A unipolar electrical conductor can have an outer diameter of about 150 microns or even smaller. The buffered fibers support conduction of high intensity electrical pulses as required for internal or external defibrillators, or other biomedical applications, as well as non-medical applications. Defibrillation pulses can be transmitted through less cross-sectional area of metal in the subject fine wire conductor than would be the case with conventional solid core metal wires. Multiple such coated fibers can act as a single conductor. An outer protective sheath of a flexible polymer material can be included.

This application claims the benefit of priority to U.S. Provisional Application No. 61/274,457, filed Aug. 18, 2009. This application also claims the benefit of and is a continuation of U.S. application Ser. No. 12/806,743, and claims the benefit of and is a continuation in part of U.S. application Ser. No. 12/156,129, filed May 28, 2008 and Ser. No. 12/590,851, filed Nov. 12, 2009. All of the above applications are incorporated herein by reference.

### BACKGROUND OF THE INVENTION

This invention concerns a durable small gauge electrical conductor suitable for use in delivery of high intensity energy pulses such as might be required for biomedical applications. The durable fine wire conductor delivers high intensity energy pulses, e.g. 30-35 joules over about 2.5 msec or less, from an electrical pulsing device, typically a capacative discharge device. These biomedical applications may include external and internal cardiac defibrillators (ECDs, ICDs), as well as neurological blocks for pain/sensory or motor control mitigation. Application of the electrical conductor of this patent application may also be towards various military and civilian non-medical roles such as might be encountered in aviation, ground transportation, boats or ships, and aerospace.

As far as medical applications are concerned, active implantable devices as represented by cardiac defibrillation and pacing, have become a well-tested and effective means of maintaining heart function for patients with various heart conditions. Generally pacing is done from a control unit placed under but near the skin surface for access and communications with the physician controller when needed. Leads are routed from the controller to the heart probes to provide power for pacing and data from the probes to the controller. Probes are generally routed into the heart through the right, low pressure, side of the heart. No left, high pressure, heart access through the heart wall has been successful. For access to the left side of the heart, lead wires are generally routed from the right side of the heart through the coronary sinus and into veins draining the left side of the heart. This access path has several drawbacks; the placement of the probes is limited to areas covered by veins, and the leads occlude a significant fraction of the vein cross section and the number of probes is limited to 1 or 2.

Defibrillation is similar to pacing in that an implantable power source with associated leads are implanted in the heart. The power source also has sensing capability through the leads for recognizing aberrant heart rhythm. When such a condition is encountered, a high intensity electrical pulse is sent through a lead to convert the heart to normal rhythm.

Over 650,000 pacemakers are implanted in patients annually worldwide, including over 280,000 in the United States. Over 3.5 million people in the developed world have implanted pacemakers. Another approximately 900,000 have an ICD or cardiac resynchronization therapy (CRT) device. The pacemakers involve an average of about 1.4 implanted conductive leads, and the ICD and CRT devices use on average about 2.5 leads. These leads are necessarily implanted through tortuous pathways in the hostile environment of the human body. They are subjected to repeated flexing due to beating of the heart and the muscular movements associated with that beating, and also due to other movements in the upper body of the patient, movements that involve the pathway from the pacemaker to the heart. This can subject the implanted leads, at a series of points along their length, through tens of millions of iterations per year of flexing and unflexing, hundreds of millions over a desired lead lifetime. Previously available wire leads have not withstood these repeated flexings over long periods of time, and many have experienced failure due to the fatigue of repeated bending.

Neurostimulation refers to a therapy in which electrical stimulation is delivered to the spinal cord or targeted peripheral nerve in order to block neurosensation. Both low-voltage applications and high intensity applications at short durations are known. The invention of this provisional application is most suited towards high intensity, short duration stimulation. Neurostimulation has application for numerous debilitating conditions, including treatment-resistant depression, epilepsy, gastroparesis, hearing loss, incontinence, chronic, untreatable pain, Parkinson's disease, essential tremor and dystonia. Other applications where neurostimulation holds promise include Alzheimer's disease, blindness, chronic migraines, morbid obesity, obsessive-compulsive disorder, paralysis, sleep apnea, stroke, and severe tinnitus.

Today's pacing leads manufactured by St. Jude, Medtronic, Greatbatch, Oscor Medical and Boston Scientific are typically referred to as multifilar, consisting of two or more wire coils that are wound in parallel together around a central axis in a spiral manner. This construction technique helps to reduce impedance in the conductor, and builds redundancy into the lead in case of breakage. The filar winding changes the overall stress vector in the conductor body from a bending stress in a straight wire to a torsion stress in a curved cylindrical wire perpendicular to lead axis. A straight wire can be put in overall tension, leading to fatigue failure, whereas a filar wound cannot. However, the bulk of the wire and the need to coil or twist the wires to reduce stress, limit the ability to produce smaller diameter leads.

Modern day pacemakers are capable of responding to changes in physical exertion level of patients. To accomplish this, artificial sensors are implanted which enable a feedback loop for adjusting pacemaker stimulation algorithms. As a result of these sensors, improved exertional tolerance can be achieved. Generally, sensors transmit signals through an electrical conductor which may be synonymous with pacemaker leads that enable cardiac electrostimulation. In fact, the pacemaker electrodes can serve the dual functions of stimulation and sensing.

The ideal characteristics of an electrical conductor for non-medical applications depend on the exact nature of the intended use. Electrical conductors used in benign applications such as providing electrical power throughout a building may consist of a simple copper conductor encased in polymeric insulation. Other non-medical applications may require that electrical conductors function with a great degree of precision and integrity in hostile environments, posing challenges to electrical conductor design that are shared with implantable medical devices. For instance, electrical conductors deployed in environments where the conductor is exposed to repetitive motion may result in fatigue failure to the conductor, not unlike what can occur with pacemaker or defibrillator leads. Non-medical electrical conductors may also be required to operate in wet conditions, which require that insulation be incorporated on the conductor to protect from direct contact with water, not unlike electrical leads of implanted medical devices.

In addition to these similarities, electrical conductors for non-medical applications may also be called upon to operate under extremes of temperature (hot and cold), chronic vibration, sunlight exposure, vacuum, or other environmental factors. These electrical conductors may also need to operate under conditions in which minimization of size and weight are required in ways that are not met by currently available electrical conductors.

It is the object of the invention described herein to a novel electrical lead construction suitable for use in implantable electrostimulation medical devices, as well as a wide spectrum of non-medical applications, where currently available electrical conductors are less than ideal for use in extreme environments encountered by the conductors. The invention is specifically directed towards a durable small gauge electrical lead capable of transmitting high intensity pulsatile stimulation for medical and non-medical applications.

### SUMMARY OF THE INVENTION

In the invention of this patent application, a flexible and durable fine wire electrical conductor, termed a lead, can be connected to a pacemaker, ICD, CRT or other cardiac or non-cardiac pulse generator, as well as non-medical devices. The electrical conductor used to fabricate a lead is formed from a drawn silica, glass, or sapphire crystalline quartz fiber core, herein referred to collectively as a glass fiber, with a conductive metal buffer cladding on the core. Alternatively, a polymer fiber core, or other suitable core material such as carbon nanotube fiber, can be used under conditions in which the physical/mechanical characteristics of fatigue-resistant glass fiber are not completely suitable. For instance, the fatigue-resistant characteristics associated with carbon nanotube fibers may be preferred under some circumstances. Use of non-glass fiber alternative core materials can be included in both medical and non-medical applications. For either a metallized core material of glass, polymer, or nanotube fiber, the structure can also be enhanced by incorporating a polymer coating over the metal buffer cladding, which may provide a biocompatible surface resistant to environmental stress cracking or other mechanisms of degradation associated with exposure and flexure within a biological system. In non-medical applications, the polymer coating may serve simply as an electrical insulation a function shared with leads intended for medical applications.

The outer diameter of the electrical conductor preferably is less than about 750 microns, and may be 200 microns or even as small as 50 microns. Metals employed in the buffer can include aluminum, silver, gold, platinum, titanium, tantalum, gallium, or others, as well as metal alloys of which MP35N, a nickel-cobalt based alloy platinum-iridium, and gallium-indium are examples. In one embodiment the metal cladding is aluminum, silver, or gold, applied to the glass fiber core. This may include immediate application upon drawing the fiber, or may involve application of metal to a pre-formed glass fiber by one of several processes including chemical or physical vapor deposition, or electroplating. Metallization of the glass fiber provides a protective hermetic seal over the fiber surface. Alternatively, the glass fiber can be hermetically sealed with carbon or polymer following drawing of the fiber, the surface of which can then be metallized by one of the processes previously mentioned. This embodiment is further detailed below.

For applications in which delivery of high voltage or current is needed, multiple fibers can be used in parallel. Alternatively, the glass fiber can be fabricated as a dielectric with a metal wire in the center of the glass fiber core as one electrical conductor, and a metallic buffer layer applied on the outside of the glass fiber core, both protecting the fiber and acting as a coaxial second conductor or ground return.

In an additional embodiment, a further layer of silica, glass, etc. (as above) covers the metallic cladding, with a further electrically conductive buffer covering that dielectric layer. This embodiment may be with or without a center wire in the inner fiber. These silica, glass, etc. layers and buffer coatings can be continued for several more layers to produce a multiple conductor cable.

In a further embodiment the center of the fiber core is hollow to increase flexibility of a lead of a given diameter. In still a further embodiment, multiple conductors are embedded separately side-by-side in the glass fiber core, where the glass serves to electrically insulate the conductors from each other.

In an additional embodiment, an electrical conductor is composed of many smaller metal-buffered or metal wire-centered glass fibers that together provide the electrical connection. This embodiment allows for high redundancy for each connection and very high flexibility.

Additional embodiments differ from the aforementioned embodiments in that metal is not necessarily applied directly to the glass fiber. As mentioned previously, a non-metal buffer such as carbon and/or polymer may be applied directly to the glass fiber core to form a protective hermetic seal layer on the fiber. Metal can then be deposited upon the carbon and/or polymer in a subsequent step. Such a metal deposition process may conveniently take place through a batch process, or via a continuous deposition process, in which carbon- and/or polymer-coated fiber is moved continuously through a deposition chamber during the metal deposition process. Such metal deposition may be carried out by vapor deposition, electroplating especially upon an electrically conductive carbon surface, by coating with an electrically conductive ink, or by one of numerous other metal deposition processes known in the art. In the case of vapor deposition and related processes governed by line-of-sight considerations, one or more metal targets sources for vaporized metal, may be positioned within the metal deposition chamber in such a way as to insure overlap and complete 360 degree coverage of the fiber during the metal deposition process. Alternately, the fiber may be turned or rotated within the vapor deposition field to insure complete and uniform deposition. Vapor deposition processes are typically carried out in an evacuated chamber at low atmospheric pressure (approximately 1.0×10⁻⁶ torr). After evacuation is attained, the chamber is backfilled with a plasma-forming gas, typically argon, to a pressure of 2.0×10⁻³ torr. Masking may be pre-applied to the carbon and/or polymer surface to enable a patterned coating of metal on the carbon and/or polymer surface. Such a pattern may be useful for creating two or more separate electrically conductive paths along the length of the electrical conductor, thus enabling fabrication of a bipolar or multipolar conductor upon a single electrical conductor. Inherent in the concept of a metallized electrical conductor according to this invention is the ability to use more than one metal in the construction of such electrical conductors. For instance, an initial metal may be deposited on the basis of superior adhesion to the carbon and/or polymer underlayment. One or more additional metals or metal alloys could then be deposited on the first metal. Intent of the second metal would be to serve as the primary conductive material for carrying electrical current.

The completed metallized electrical conductor may then be conveniently coated with a thin polymeric material, (polytetrafluoroethylene (PTFE) for example) to provide insulation and/or lubriciousness. Also, polyurethane or silicone or other insulative polymers may conveniently be used as jacketing material, providing biocompatibility and protection from the external environment. A coaxial iteration of this embodiment incorporating two independent electrical conductors may be constructed in which a metal electrical conductor is embedded within the central glass or silica core, with the second conductor being applied to the carbon and/or polymer buffer residing on the outer surface of the glass or silica core.

In an additional embodiment of metal cladding for the glass fiber, temporary sealing materials may be applied to the glass fiber for protection. Subsequent steps carried out in a controlled environment facilitate removal of the temporary sealing materials, followed by resurfacing the fiber with metal or other material, such as polymer or carbon. Such steps enable controlled metal surfaces to be applied directly to the glass fiber, if so desired. Temporary sealing materials may consist of polymers, carbon, or metals, which are chosen ease of removal. In the case of polymers, removal may be facilitated by dissolution in appropriate solvent, heat, alteration in pH or ionic strength, or other known means of control. Carbon and metals may be removed by chemical or electrochemical etching, heating, or other known means of control.

As indicated previously, various metals or metal alloys may be suitable for employment as a permanently deposited electrical conductor of this invention. Idealized properties include excellent electrical conductivity with low electrical resistance, resistance to corrosion, or heat, which may be employed at various steps during the electrical conductor manufacturing process. Additional resistance to exposure to cold, vacuum, vibration, and cyclic bending fatigue represent desired characteristics.

Estimated metal cross sectional area for a solid metal wire, having a desired electrical resistance, may be determined theoretically from the following relationship:

R=ρ*(1/A),

where R=resistance (ohms), ρ=metal resistivity (ohms-cm), 1=conductor length (cm) and A=cross sectional area of conductor. Thus, desired resistance is equal to the product of resistivity and the quotient of length and cross-sectional area. For some applications of the electrical conductor of this invention, desired electrical resistance may be on the order of 50 ohms. Using silver as an example, resistivity is 1.63×10⁻⁶ ohms-cm. Thus, a silver conductor of approximately 1000 nm thickness would provide the desired electrical resistance for an electrical conductor of approximately 0.015 cm diameter and 80 cm length.

The electrical conductor of this invention, whether coaxial or otherwise in construction, is extremely strong and flexible. The invention contemplates cables (meaning glass fiber incorporating one or more electrical conductors) of as little as 100 to 200 micron diameter, and even smaller, down to 50 micro diameter, or as large as 750 microns or more in diameter, and even unipolar electrical conductors as small as 50 microns in diameter or even smaller. These small diameter electrical conductors have significant flexibility with an achievable bend radius of as little as 0.5 mm, to provide placement in tortuous tracts, as might be encountered in the heart in the case of pacemaker leads, or in fine electronic circuitry as might be incorporated in both medical and non-medical electrical instrumentation.

The multipolar electrical conductor representing one embodiment of this invention adapts technologies that have been developed for various disparate applications. Glass fiber is produced from a draw tower, a furnace that melts the silica or glass (or grown crystals for the sapphire and quartz) and allows the fiber to be pulled, “drawn”, vertically from the bottom of the furnace. Fibers produced in this manner have strength of over 1 Mpsi. If the drawn fiber is allowed to sit in normal atmospheric conditions for more than a few minutes, that strength will rapidly degrade to the order of 2-10 kpsi. This reduction is caused by water vapor attack on the outer silica or glass surface, causing minute cracking. Bending the silica or glass fiber causes the outside of the bend to be put into tension and the cracks to propagate across the fiber causing failure. To ensure that the fiber remains at its maximum strength, a buffer is added to fibers as they are drawn. As the fiber is drawn and cools, a plastic coating, the buffer, is applied in a continuous manner protecting the fiber within a second of being produced.

The TOW missile was developed during the 1960s as an antitank missile for the U.S. Army. The missile was launched from a shoulder mounted launcher and was guided to the target by an optical system that included a fiber spooled from the rear of the missile as it flew. The fiber had to be very strong and light to unreel several kilometers of fiber in a few seconds. Fiber optics was selected, but to further strengthen the fiber and protect it from damage, the plastic buffer was replaced with a metal buffer. The metal buffer used at that time was aluminum, but systems to coat fibers with gold and other metals have since been developed. The patents for the metal buffer technology covered a wide range of metals and alloys and were issued to Hughes in 1983 (U.S. Pat. No. 4,407,561 and U.S. Pat. No. 4,418,984).

The concept of using glass fibers incorporating optical capabilities in a coaxial construction was developed for micro miniature x-ray sources by Xoft, Inc., Photoelectron Corporation and others. See U.S. Pat. Nos. 6,319,188 and 6,195,411. These fibers were used because they provided high flexibility, high voltage hold-off and direct connection to the x-ray source without a joint between the x-ray source and the HV power supply. The standard available optical-capable fiber did not include a central electrical conductor. To include a wire in the center of the fiber, the wire must be drawn with the silica, glass, etc. in the draw tower. For optical applications, to ensure that any optical energy launched into the fiber is not absorbed at the core wire interface, an additional lower index silica or glass cladding is provided between the core and the wire. All this is known prior practice. The electrically conductive glass fiber of the invention of this application does not require an extra silica or glass cladding for use with non-optical electrical conduction.

Alternative methods of producing a coaxial electrically conductive glass fiber include drawing a core fiber, coating that core with a metal buffer and drawing additional silica or glass over the assembly and cladding that final assembly with an additional metal buffer. Fibers can be pulled with a hole in the center as well, increasing flexibility; hole diameter can vary. In one embodiment one or more wires can be put inside the hole through a fiber. The fiber can be redrawn to engage the wire if desired.

Additional embodiments can also be defined, where the glass fiber, either solid core or hollow, can act as the strength member and dual electrical conductors can be placed outside the fiber system and separated by plastic or polymer insulators. Fatigue of metals and plastics after millions of small deflection stresses is one of the life-limiting aspects of conventional wire constructions of conventional pacing leads and other conductors used for both medical and non-medical applications. Silica, glass, etc. fibers protected with robust buffer systems will not exhibit fatigue. Fatigue in silica or glass is caused by propagation of cracks, which are present at low levels in typical silica or glass fibers as produced for standard communication purposes. Typically they exhibit a few surface flaws per kilometer of fiber. Therefore silica or glass fiber coax cables make ideal pacing leads; small diameter, low mass, highly flexible, robust and with very long service life. Such attributes are also what make the glass fiber electrical conductor of the invention attractive for use in non-medical applications. Sophisticated electrical equipment represent a hallmark of the modern military force, and a small profile, lightweight, and durable electrical conductor, resistant to breakdown from heat, cold, environmental contamination, and/or solar exposure would have immediate usefulness across many possible scenarios. Examples include, but are not limited to, soldier interconnects, avionics, command and control, weapons, communication, data acquisition, and imaging. Multiple civilian applications can also be identified where the unique features of the invention can be applied. Examples include all motorized modes of transportation where a light durable electrical conductor is desired.

One method according to the invention for testing fibers intended for use in electrical conductors is to stretch a long segment until it breaks; the weakest point in the fiber will break first. If the fiber meets some minimal standard for tensile strength, then the entire fiber meets some strength minimum and flaws will not exist up to some level. If the fiber does break, the remaining pieces can be similarly tested. As this is repeated the limits at which the fiber will break will continue to climb allowing selection of extremely flaw free sections of fiber. This will further enhance the ability of the fiber to resist failure due to repeated stress cycling. This is a type of fiber “proofing”, but proofing as previously known was for lot testing rather than for selections of sections of highest strength from a fiber. Pursuant to the invention fibers for use in the electrical conductors are proofed to at least about 90% of the intrinsic strength value of the material, or more broadly, at least about 75%.

The glass/silica electrical conductor of the invention, as envisioned for implantable electrostimulation medical devices, is compatible with drug/steroid elution for controlling fibrosis adjacent to a terminal electrode, which is a known technique used with conventional pacing leads for controlling impedance and thus battery life. For example, a biodegradable polymer can be positioned on the distal end of a lead at the terminal electrode, with the polymer containing the eluting drug.

It is among the objects of the invention to improve the durability, lifetime flexibility and versatility of wire leads for implantable electrostimulation medical devices such as pacemakers, ICDs, CRTs and other cardiac high-energy pulse generators, as well as electrostimulation or sensing leads for other therapeutic purposes within the body. It is also an object of the invention to reduce the weight and size associated with an electrical conductor over those of previously available electrical conductors, for applications where such characteristics are desired. Applications including medical and non-medical, military and civilian, terrestrial and aeronautic are all anticipated. These and other objects, advantages and features of the invention will be apparent from the following description of embodiments, considered along with the accompanying drawings.

### DESCRIPTION OF THE DRAWINGS

FIG. 1 is a schematic view partially cut away, showing a human heart, and indicating a path of pacemaker or other cardiac electrostimulation leads in accordance with conventional practice.

FIG. 2 is a schematic drawing in perspective showing one embodiment of an electrical conductor as intended for use in extreme environmental conditions.

FIG. 3 is a similar view showing another embodiment of an electrical conductor.

FIG. 4 is a view showing a further embodiment of an electrical conductor.

FIG. 5 is a view showing another embodiment of an electrical conductor.

FIG. 6 is a view showing an embodiment with twisted or braided multiple conductors.

FIG. 7 is a schematic perspective view showing another form of fine wire electrical conductor

FIG. 8 is a sectional view showing a connector at an end of an electrical conductor of the invention.

FIG. 9 is a schematic drawing in perspective showing another embodiment of an electrical conductor.

FIG. 10 is a similar view showing another embodiment of an electrical conductor.

FIG. 11 is a view showing a further embodiment of an electrical conductor.

FIG. 12 is a view showing electrical conductor with multiple conductors.

FIG. 13 is a depiction of a mechanism and movement path for continuous processing of an electrical conductor for metal deposition.

FIG. 14A is a cross sectional view of a possible patterned metal deposition on an electrical conductor(s).

FIG. 14B is a cross sectional view of a possible patterned metal deposition on an electrical conductor(s).

FIG. 14C is a cross sectional view of a possible patterned metal deposition on an electrical conductor(s).

FIG. 15A is a cross sectional view of a possible electrical conductor having a single continuous metal electrical conductor.

FIG. 15B is a cross sectional view of a possible electrical conductor having a single continuous metal electrical conductor.

FIG. 16A is a schematic perspective showing an electrode deposition pattern made up of one or two metal electrical conductors. The electrical conductor may also incorporate a metal conductor at the center of the glass fiber core, resulting in a coaxial construction.

FIG. 16B is a cross sectional view showing an electrode deposition pattern made up of one or two metal electrical conductors. The electrical conductor may also incorporate a metal conductor at the center of the glass fiber core, resulting in a coaxial construction.

FIG. 16C is a schematic perspective showing an electrode deposition pattern made up of one or two metal electrical conductors. The electrical conductor may also incorporate a metal conductor at the center of the glass fiber core, resulting in a coaxial construction.

FIG. 16D is a cross sectional view showing an electrode deposition pattern made up of one or two metal electrical conductors on a electrical conductor. The electrical conductor may also incorporate a metal conductor at the center of the glass fiber core, resulting in a coaxial construction.

### DESCRIPTION OF PREFERRED EMBODIMENTS

The invention encompasses electrical conductors for all implantable electrostimulation and sensing devices having implanted wire leads, as well as non-medical applications where light weight and durability are important characteristics contributing to the performance of the electrical conductor, especially in extreme environmental conditions. Also necessary is a capability of the lead to withstand physical stresses imposed by passage of high intensity electrical pulses along the conductor.

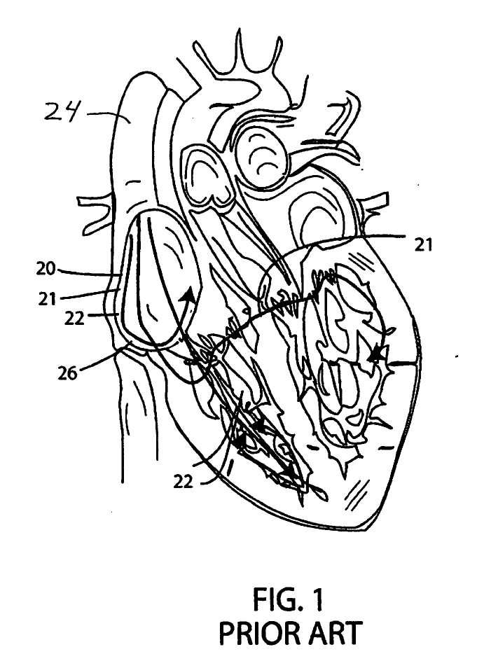

FIG. 1 shows schematically a human heart with some walls cut away. In FIG. 1 pacing leads are shown following a conventional path into the heart, and into the cardiac veins of the left ventricle, as has been typical of conventional practice and which, with some exceptions, is the basic path of leads of this invention.

In typical conventional practice, conductive leads 20, 21 and 22 are introduced into the heart through the superior vena cava 24, brought into the vena cava via subclavian or cephalic vein access points. For the right side of the heart, separate conventional pacing electrodes, as well as separate electrodes for biventricular pacing are normally routed into right ventricle, as well as the right atrium. For the left ventricle, typically a wire lead 21 would be brought from the right atrium 26 into the coronary sinus, and from there the leads are extended out into one or more coronary veins adjacent to the surface of the left side of the heart. The leads are not introduced directly into the interior of the left ventricle, which is the high-pressure chamber.

Pursuant to the invention the routing of silica/glass fiber leads can be essentially the same as with conventional leads. An important difference is that the silica/glass lead, being much smaller diameter than conventional leads, can be positioned deeper and more distally (also “retrograde” to normal blood flow toward the coronary sinus) within the target coronary vein. The coronary sinus/coronary vein architecture can be a relatively tortuous path, such that the physician will have an easier time manipulating a smaller diameter, flexible lead into the desired position within the coronary vein than for a larger diameter lead. Also, as a lead is manipulated deeper (more distally) within the coronary vein, the diameter of the vein becomes progressively narrowed. Thus, a smaller diameter lead can be placed deeper than a larger diameter lead. One theoretical reason why it is useful to place the terminal electrode of the lead in the deeper/distal/narrower portion of the coronary vein is that that portion of the vein apparently lies closer to myocardium. Thus, the cardiac muscle can perhaps be stimulated with less energy use when the electrode is closer to intimate contact with muscle overlying the coronary vein.

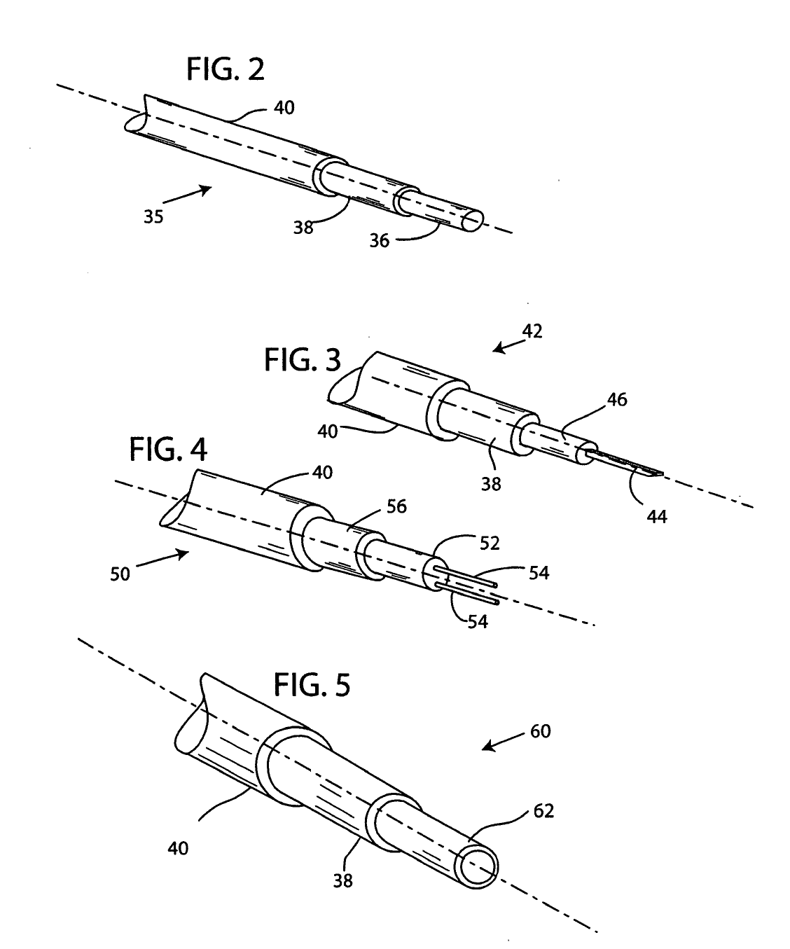

FIG. 2 is a simple schematic showing one preferred embodiment of an implantable electrical conductor 35 pursuant to the invention. In this form the electrical conductor 35 is unipolar. It has a drawn fiber core 36 of glass, silica, sapphire or crystalline quartz (“glass/silica” or “silica/glass”) with a conductive metal buffer 38 over the fiber core. As discussed above, in this embodiment, the buffer 38 is coated directly onto the fiber immediately upon drawing of the fiber, to preserve the strength of the fiber, protecting it from environmental elements such as atmospheric moisture that can attack the glass/silica surface and introduce fine cracking. Aluminum, silver, or gold are preferred metals to form the buffer 38 because of abilities to achieve hermetic bonding with the silica or glass surface, although other suitable metals or metal alloys can be used. The metal or metal alloy buffer can be about 20 microns thick, or 5 microns thick or even thinner. The wire lead 35 will make separate electrical connections (not shown) at either end.

FIG. 2 also shows a polymer coating 40 as an outer buffer. This buffer is also added very soon after drawing, and is applied after the metal buffer 38 in a continuous manner. The plastic outer buffer coating 40 may be biocompatible for intended medical uses. Likewise, biocompatibility is likely not required for most non-medical applications. Other desired characteristics of the polymer layer include being impervious to sunlight, dust, water, and exposure to cold or heat, within the intended range of operating temperatures. As discussed further below, a further metal buffer can be added over the metal buffer 38 prior to addition of the plastic coating. This can be a coating of gold or platinum, both of which are biocompatible, or some other metal or metal alloy, such as gallium, gallium-indium, or MP35N. The plastic buffer 40 adds a further protective layer.

FIG. 3 shows a modified fine wire electrical conductor 42, which has a metal conductor 44 as a center element. Here, the pure silica/glass fiber core 46 is drawn over the metal conductor 44.

The process is well known, with a hollow glass/silica fiber first produced, then a metal conductive wire placed through the hole in the fiber and the glass/silica fiber drawn down against the wire. A conductive metal buffer is shown at 38 over the fiber, having been applied immediately on drawing of the conductor-containing fiber 46. An outer buffer coating of polymer material is shown at 40, which may or may not be biocompatible, depending on the service environment of the electrical conductor.

FIG. 4 is a similar view, but in this case showing a fine wire electrical conductor 50 formed of a glass/silica fiber core 52 formed over two metal conductors 54. The wire is pre-coated with a thin layer of glass before being co-drawn with fiber. A metal buffer coating 56 surrounds the silica fiber 52, protecting the fiber from deterioration as noted above, and this can serve as a third electrically conducting element if desired. Again, an outer polymer buffer 40 provides an outer protective jacket and may be biocompatible.

In FIG. 5 is shown another embodiment of a fine wire electrical conductor 60 of the invention. In this case the glass/silica fiber core 62 is hollow, allowing for better flexibility of the electrical conductor of a given diameter, and the electrical conductor construction is otherwise similar to that of FIG. 2.

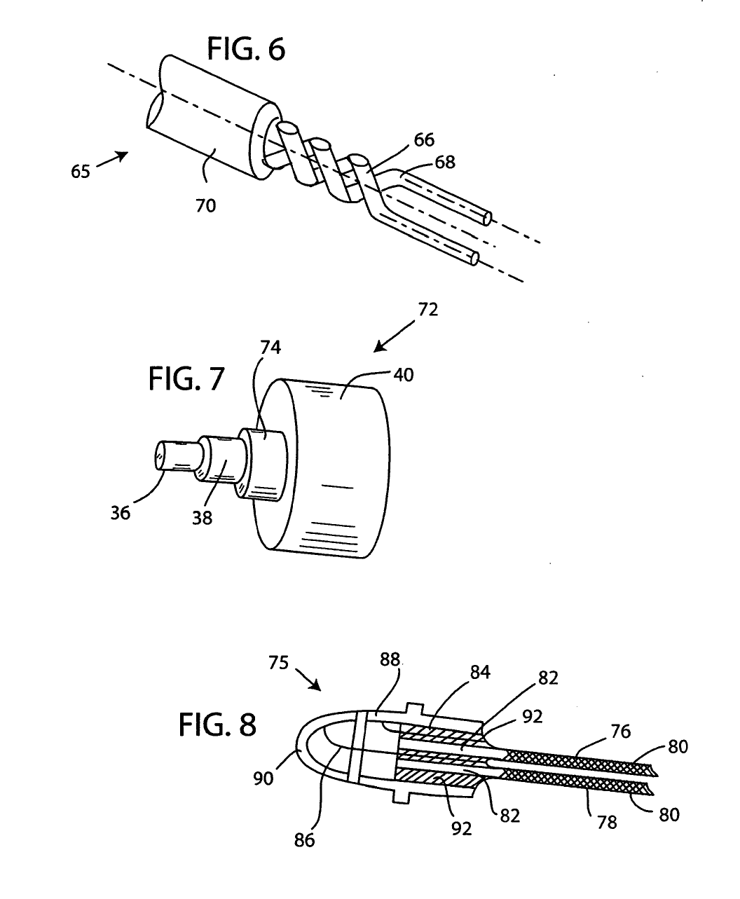

FIG. 6 shows a modified embodiment of a fine wire electrical conductor 65 which has multiple glass/silica fiber electrically conductive components 66 and 68 in a helical interengagement, twisted together. Each electrically conductive component 66, 68 comprise a glass/silica fiber conductor which can be similar to what is shown in FIG. 2, with or without a polymer buffer coating 40, or each could be constructed in a manner similar to FIG. 3, with or without a plastic buffer coating. Although two such fiber electrical conductors are shown, three or more could be included. The glass/silica fiber cores provide for strength and small-radius bending of the helical electrically conductive components 66, 68, and this type of braiding or helical twisted arrangement is known in the field of electrostimulation medical devices, and is intended for absorbing stretching, compression, or bending in a flexible manner. An outer polymer coating 70 protects the assembled electrically conductive components and provides biocompatibility, if so desired. The electrically conductive components 66, 68 themselves can have a single metal cladding, consisting of aluminum, silver, or gold, or other metal as their outer layer, or they can have one or more further layers of metal, glass and polymer.

FIG. 7 shows a section of an electrical conductor 72, which is similar to that of FIG. 2, with a silica core 36 and an initial metal cladding 38, but with a further metal cladding 74 over the inner metal cladding. Use of two dissimilar metals in direct physical contact is intended to take advantage of a first metal having desired bonding characteristics to the silica or glass core, with a second metal having desired electrical conductivity characteristics, and having durable bonding behavior on the first metal. The outer layer of polymer material is shown at 40.

FIG. 8 shows one example of a terminal or connector 75 of the invention, coupled to one end of two silica/glass fiber electrical conductors 76 and 78, each of which may be formed as described above, with a conductive buffer 80 on the exterior of each. FIG. 8 represents a biaxial pair of electrical conductors making electrical connection with two separate electrically conducting components of a terminal connector, respectively. In the type of connector 75 shown in FIG. 8, the glass/silica fibers 82 of each of the separate electrical conductors 76 and 78 extend into the connector as shown. A high temperature wire 84, 86, such as fabricated of Kovar having thermal expansion characteristics similar to glass, is welded to each of the conductive buffer claddings 80 of the two electrical conductors 76 and 78, respectively. This welded connection is made essentially outside the terminal 75, to the right as viewed in FIG. 8, where the cladding 80 on the terminal ends of the electrical conductors will not be oxidized or rendered non-conductive by the formation of the terminal or connector. These wires are connected to respective ones of two electrically isolated sections 88 and 90 of the terminal. The two sections 88 and 90 are of conductive metal and are adapted to plug into a socket formed to receive this connector 75. Alternatively (not shown), a uniaxial connection between a single electrical conductor 76, 78 and a terminal connector with incorporating a single electrically conductive element may be envisioned.

Again referring to FIG. 8, inside the connector 75, the fibers and conductive wires 84, 86 are sealed within the connector portion 88 using a relatively low temperature glass 92. The connector wires 84, 86, if of material such as Kovar, will not deteriorate even if a high temperature glass is used for sealing. The glass seal 92 does not extend over the weld connection from the wires 84, 86 to the buffer 80 on each of the electrical conductive elements 76 and 78. These weld connections and the unprotected portions of the wires 84, 86 need to be protected, covered by an appropriate material at the back end of the connector 75, where the two electrical conductor components 76 and 78 emerge from the connector. They can be covered by a polymer, or more preferably a metal buffer can be applied to each individual wire/buffer 80 connection. This could be done before or after sealing with the glass seal 92. If a high temperature transition metal such as platinum is used for this purpose, the connection between the Kovar wire and the fiber could be protected from a high temperature glass seal 92, assuming a high temperature material is used here, in the case where the glass seal 92 is applied after the Kovar wire connection is made to the fiber. In this way a hermetic seal is achieved. As indicated, analogous terminal connectors can be formed on unipolar, single-fiber electrical conductors or on bipolar electrical conductors having an exterior conductive buffer and an interior wire.

In an alternate embodiment to the details represented in FIG. 8, the process used to apply metal to the glass/silica fiber core may be modified to increase the thickness of metal coating at either the proximal end, the distal end, or both ends in such a way that the length and thickness of enhanced metal coating facilitates connection with the connector at the proximal end, and/or connection with an electrode at the distal end. Alternatively, metal may be deposited at the distal end to actually produce an integral electrode of the deposited metal of desired length and diameter. In another iteration, base metal of the same composition as the conductor may be applied at the position of the electrode, of a given length and thickness, to which a second metal is then applied by similar or different processes to produce the final electrode. The second metal is chosen on the basis of superior electrical conductivity and low resistivity, as compared to the first metal which may be chosen both for the sake of good electrical conductivity as well as good adhesion to base core material. Electrodes and connectors may be attached by welding, adhesive bonding using electrically conductive adhesives, or mechanical crimping.

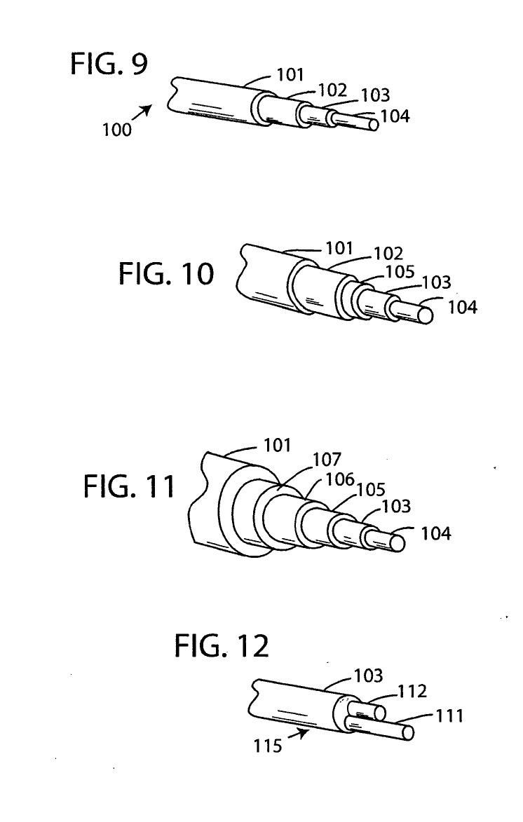

FIG. 9 represents a fine wire lead 100 in which metal 102 is deposited directly onto carbon hermetic seal material 103 overlying the glass/silica fiber core 104. Upon the metal layer, a polymer-based insulator 101 is applied. This insulator may be Teflon, or other lubricious polymer coating that is ideally resistant to mechanical- or friction-based wear or degradation with resultant cracking or physical loss from the fine wire lead. The carbon layer is relatively thin in profile, consisting generally of 10-1000 Angstroms in thickness. The metal layer may be on the order of 0.1-10 microns in thickness. The outer insulator does not require significant thickness for low-current applications as envisioned by this invention and thus may be 1-10 microns in thickness.

The carbon hermetic seal layer 103 can be deposited onto the glass/silica fiber core by any of several known techniques, such as plasma enhanced chemical vapor deposition using methane and hydrogen as the precursor gases. As reported in “Effects of annealing on the properties of hermetically carbon-coated optical fibers prepared by plasma enhanced chemical vapor deposition method”, Opt. Eng., Vol. 46, 035008 (2007); dol: 10.1117/1.2716015, Mar. 21, 2007, incorporated herein by reference, annealing temperature is important in this process. A related iteration (not shown) incorporates a polymer layer in direct contact with the glass core 104, as a substitution for the carbon hermetic seal material 103. As an alternate to the lubricious polymer insulator 101, a polymer insulator with optimized biocompatibility such as polyurethane or silicone may be utilized.

FIG. 10 is similar to the fine wire lead depicted FIG. 9, with the incorporation of an additional polymer layer 105. This polymer resides between the metal hermetic seal material 103, and the conductive metal layer 102. This polymer layer 105 can provide protection to the carbon layer 103, as well as an improved bonding surface for metal deposition. The layer 103 could alternatively be a metal layer.

FIG. 11 is also similar to the fine wire lead in FIG. 9. In this case two separate metals 106 and 107 are deposited on a polymer clad material 105. The two separate metals can serve different functions, including optimization of tensile strength, crack resistance, electrical conductivity, and adhesiveness to underlying materials. Related iterations (not shown) being similar to FIGS. 9 and 10, would include more than two metal layers, and/or a carbon hermetic seal material 103 but no polymer cladding 105 or a polymer clad material 105 but no carbon layer 103.

FIG. 12 represents a fine wire lead with two conductive glass fibers 111 and 112, where each individual glass fiber reflects construction details according to previous figures, including individual glass/silica cores, hermetic seal materials of carbon and/or polymer, metal deposition, and polymer insulation. A single multiconductor fine wire lead 115 is thus fabricated by jacketing two or more conductive glass fibers within a single outer polymer jacket 103. This jacket is conveniently fabricated of biocompatible material such as polyurethane or silicone.

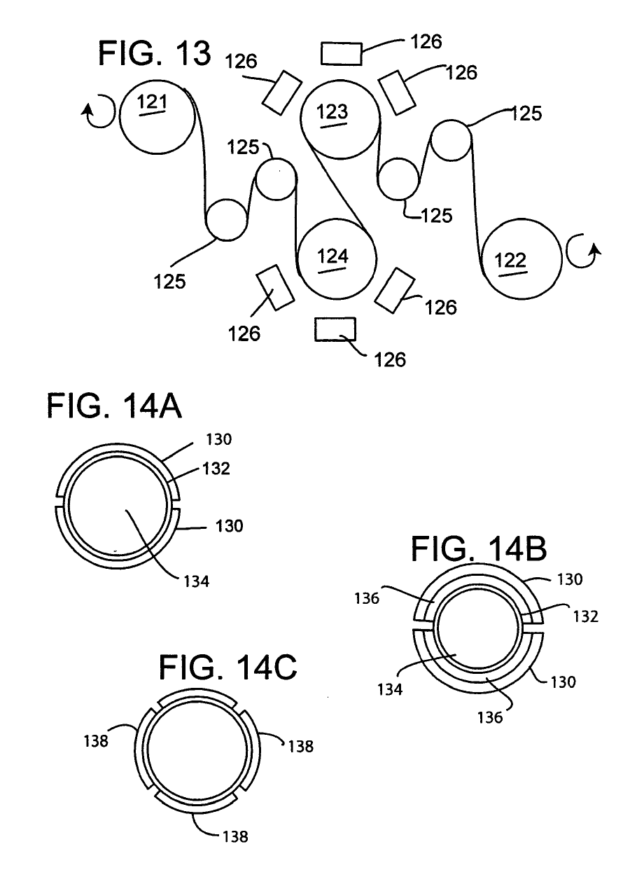

FIG. 13 is a depiction of a mechanism and movement path for continuous processing of a fine wire lead for metal deposition. The metal deposition process is designed to take place within a vacuum chamber in which gas composition and pressure may be controlled. Various motor-driven rollers 121-125 are set to provide directionality, tension, positioning, and duration of glass fiber substrate within the metal deposition field. At 121 is a feed roller, and at 122 a take-up roller. Chill drums are at 123 and 124. Metal source targets 126 are positioned within the chamber to provide adequate coverage of the glass substrate. Actual position of the metal source targets may or may not be directly adjacent to rollers within the chamber.

FIGS. 14A-14C are a series of cross sectional views of several possible patterned metal depositions on fine wire leads. These depositions are conveniently carried out using masks in order to produce two or more independent electrically conductive paths down the length of the fine wire lead. Depicted are patterns involving two or four electrically conductive paths, made up of a single metal deposition, but may also represent deposition of two separate metals as in FIG. 14B. FIG. 14A shows two metal segments 130 in a single layer over a polymer buffer cladding 132 on a central core 134. In FIG. 14B two metal layers 130 and 136 form the two segments. In FIG. 14C four different metal segments 138 are shown, in a single layer.

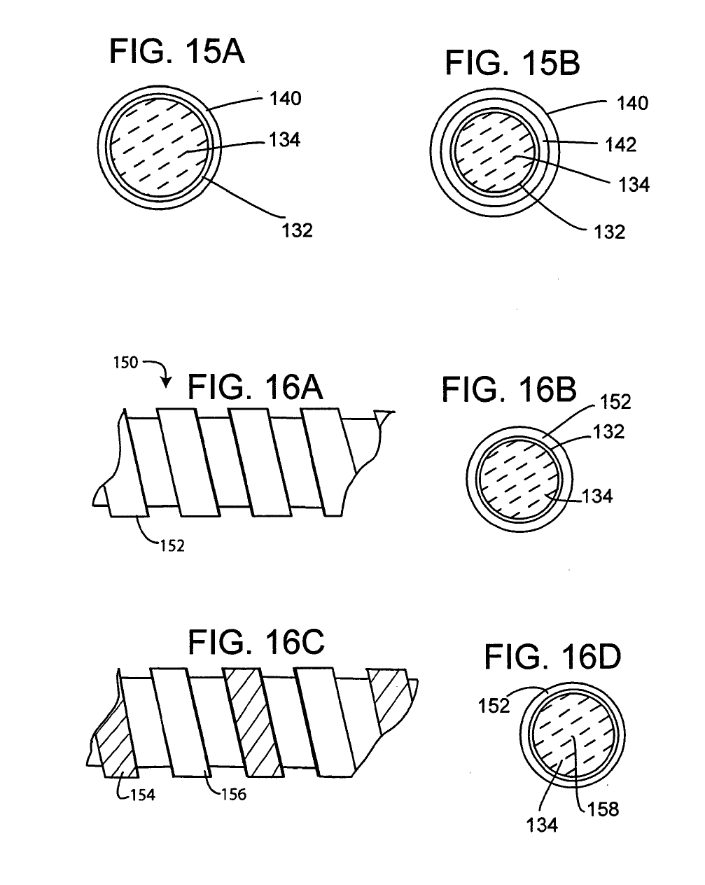

FIGS. 15A and 15B show cross sectional views of two possible fine wire leads having a single continuous metal electrical conductor 140. The two cross sections differ in that one (FIG. 15A) incorporates a single metal 140 in the conductor, while the other (FIG. 15B) depicts a cross section in which two separate metals 140 and 142 are incorporated in a single electrical conductor. In both cases the metal is over a polymer buffer cladding 132, which covers a fine glass fiber core 134, preferably a thin hermetic or non-hermetic coated glass core. The two metals in FIG. 15B differ on the basis of fatigue resistance, electrical resistance, as well as other properties which might include heat conductance, melting point, and adhesion to underlying materials. The inner layer 142 can be a lower electrical resistance metal, while the outer layer 140 can be a high mechanical fatigue resistance metal.

FIGS. 16A-16D are a series of schematic side and cross sectional views showing an electrode deposition pattern made up of one or two metal electrical conductors on a fine wire lead. In FIG. 16A a pattern 150 has a single metal 152 coated in a helical pattern on a glass fiber surface. FIG. 16B shows the lead in cross section and shows the glass core 134 can have a polymer buffer cladding 132. Masking may be used to enable patterned conductors as helical paths as shown, or other patterns. FIG. 16C shows a pattern with two isolated conductors 154 and 156 in helical configuration. Coaxial leads may also be constructed as depicted in the cross section of FIG. 16D, in which a metal lead 158 is deposited in the center of the glass core 134.

The following, including Appendices 1 and 2, relates to the conductors described above as used to deliver high intensity energy pulses such as for cardiac defibrillators and similar applications as described above.

As described earlier in this application, the theoretical performance of a solid wire conductor with respect to resistance can be calculated. Such a relationship should also apply towards the construction of this invention in which a metal layer is placed over a non-conductive glass fiber core. However, when the conductor is used to transmit high intensity electrical energy along the conductor, heating can become an issue. Resistant heating of a conductor can cause it to fail, due to melting and/or breakage of the electrical connection at one or multiple points along the length of the conductor. The electrical intensity necessary to cause such failure, referred to as the fuse point, or fuse current, can be estimated by calculation.

It has been found that the invention of this application, namely an electrical conductor based on a metal coated glass/silica core fiber, allows a higher intensity pulsatile electrical load to be transmitted through an electrical conductor than would be estimated for a solid wire of the same metal, and of the same metal cross-sectional area. Thus, for a multifilar lead construction, a lower number of filars are needed to supply the necessary cross-sectional area of metal to carry high intensity electrical pulses, than would be predicted from theory for a solid wire of the same metal.

See Appendix 1 for details on the theoretical approach for estimating the number of filars required to support defibrillation. See Appendix 2 for actual bench test results for representative filars.

The actual test results indicate that a lower number of filars are necessary to support defibrillation than predicted from theory. Probable reasoning includes several factors thought not to be fully appreciated prior to this invention.

The metal on a glass/silica core filar with metal coating is organized differently than for a solid core metal wire of equivalent cross-sectional area. The metal coating has a much greater surface area, including the metal surfaces facing both externally, and internally. This enables much greater heat dissipation than afforded by a solid metal wire.

The glass/silica core acts as a heat sink, enabling rapid heat transfer from the thin metal coating. The solid core metal wire on the other hand has no available internal heat sink.

A defibrillation pulse represents a capacitor discharge with exponential decay, characterized by an intense initial discharge followed by decreasing intensity throughout the remainder of the pulse width. This is unlike a square wave pulse as anticipated by the Onerdonk equation, or continuous current delivery as anticipated by the Preece equation.

### Appendix 1

### Defibrillation Lead Theoretical Estimation

Defibrillation therapy treats dangerously fast heart rhythms. When a CRT-D shocks the heart back to normal rhythm, it uses higher energy. The energy used to restart the heart is estimated to be 30-35 Joules. Based on this starting figure, we can calculate the amount of amperage needed to be carried by the lead.

### Per the ISO Standard:

A simplified defibrillation circuit relies on the discharge of a 200 μF capacitor to deliver energy to the heart. Per Annex B of ISO 11318:1993 (DF-1), the test configuration simulates a clinical situation where a 1000V defibrillation output from a 200 μF capacitor is delivered to a patient presenting a system resistance of between 20Ω and 25Ω. 20Ω system resistance is at the extreme low end of impendence seen clinically, and results in the highest current. This test represents a safety factor of at least 2.

### Per Applied Limits of an Actual Defibrillation Pulse:

Looking at the application limits, a defibrillation pulse delivers between 30 J and 35 J energy to the heart over a 25 msec time period. Using the capacitor equation:

E=1/2CV² or 30J=1/2(200 μF)V²

Voltage can be estimated at 550 volts. Setting the system resistance is 25Ω, the amperage is calculated as

V=I*R or 550=I*(25)

I=22 amps

To estimate the number of glass/silica core filars having thin metal coatings that would be required to carry the current pulse for a defibrillation, one relies on calculating the fuse point, or fuse current. A fuse is a circuit element designed to melt when the current exceeds some limit, thereby opening the circuit.

### Preece Equation

The basic design equation for fuses is the Preece equation (W. H. Preece, Royal Soc. Proc., London, 36, p 464, 1884) for wires in free air:

I=A*D̂1.5

where A is a constant depending on the metal. For silver, A=3200 and D is the diameter of the wire in inches. For the coated silica fiber, the cross sectional area of the metal is calculated as an annular ring and that area converted to a circular wire and the diameter used in the equation. *Exponent in the equation should be adjusted to 1.287 for silver and 1.32 for tungsten. Solving for amps:

A=3200*D^{(1.287)}

D is calculated for an 800 nm coating on a 157 micron fiber using the equation:

D=2*SQRT[(Do/2)²−(Di/2)²]

where Di=inner diameter of the coating and Do=outer diameter of the coating.

Based on the cross sectional area of the thin film, the total amperage that can be carried by the silver coating is calculated using the Preece equation to be 0.0297 amps. Dividing our overall amperage of 22, by the fuse current calculated above, estimates that 740 filars are required to carry 22 amps of current.

The above analysis is based on cross sectional area of the metal and melting temperature of the specific metal to calculate the melt temperature and failure of the wire. It also assumes a continuous current, not a pulsed current as is seen in the defibrillation application.

### Onerdonk Equation—Square Wave Pulse

The Onerdonk equation takes into account the time of a pulse as opposed to a continuous current as described above. This may be a more accurate estimate for our application.

$$I = 1973 * A * \sqrt{\frac{log [ \frac{Tmelt - Tambient}{234 - Tambient} + 1 ]}{Time * 33}}$$

### Where

Tmelt=melting temp of wire in deg C
Tambient=ambient temp in deg C
Time=melting time in seconds
Ifuse=fusing current in amps
Area=wire area in circular mils
*Circular mils is a unit of area used in the US, particularly in connection with electrical codes. It is the diameter of the wire in thousandths of an inch (mils) squared. That is, it is the area of a circle 0.001″ in diameter. (1 cmil=0.507E-3 sq mm)
Using the same cross sectional area conversion to circular mils, and calculating fuse current, the Onerdonk equation estimates the fuse current in one filar to be 0.748 amps for a 25 msec pulse. Dividing the overall amperage required (22) by 0.748 leads us to 29 filars with 800 nm of silver coating each.

### Onerdonk Equation—Exponentially Decaying Pulse

Estimating the discharge from a capacitor during a defibrillation pulse as an exponential decay function as opposed to a square wave used in the analyses above, one can use a series of rectangles to more closely approximate the total energy seen by the lead conductor.

Breaking the pulse width of 25 msec in to 10 sections, each rectangle would represent time of 2.5 msec and each subsequent rectangle would require lower peak current. The next pulse would start at the ending current of the previous pulse. Using a simple log equation to predict the beginning and end values and starting with 22 amps as the absolute peak value at the beginning of the first rectangle, the following table can be generated:

| Time (msec) | Est Current at beginning of rectangle (amp) |
|---|---|
| 0 | 22.0 |
| 2.5 | 13.3 |
| 5.0 | 8.1 |
| 7.5 | 4.9 |
| 10.0 | 3.0 |
| 12.5 | 1.8 |
| 15.0 | 1.1 |
| 17.5 | 0.7 |
| 20.0 | 0.4 |
| 22.5 | 0.2 |

Assuming the first rectangle represents the maximum energy to be carried, as long as the filars are not damaged, with all subsequent rectangles the energy would be under the maximum and irrelevant in the failure analysis. Using the Onerdonk equation and a pulse width of 2.5 msec, the number of filars needed to carry 22 amps for 2.5 msec can be calculated as 9 filars. Because the Onerdonk equation uses time as a variable, this equation more closely predicts the behavior of filar when an exponentially decaying pulse is applied.

### DISCUSSION

This analysis assumes the overall circuit resistance is 25 ohms which includes the lead and the heart. The greater the resistance represented by the lead portion of the circuit, the greater the number of filars needed to support a high intensity pulse at the 22 amp level

The ISO Standard recommends 1000V testing which is twice the amount of voltage used for the analysis. With a 1000V pulse, and 25 ohm resistance, the amperage can be calculated to be 40 amps as opposed to 22 amps. This would result in more filars required to carry the current in the lead.

The exponential decay analysis assumes that each subsequent rectangle starts at ambient temperature. Assuming the filars will heat up due to the current generating in the previous rectangle, more filars may be needed to reduce the risk of damage.

Information in this report represents an estimate of the number of filars needed to support defibrillation

Bench testing is required to verify these results.

### Appendix 2

### Max Current Capacity Testing

The purpose of this testing was to determine the current carrying capacity of a newly designed electrical conductor, namely a filar, consisting of a glass/silica core fiber, having a thin metal coating, which was initially developed for low current applications (cardiac sensing and pacing). This testing was considered critical to understanding the capacity of the initially designed filar for use in a new application, namely that of a defibrillation lead construct. The data generated indicates number of filars required to deliver the high amperage requirements of a defibrillation pulse.

Initial standard industry calculations hypothesized that a single filar would have a carrying capacity of 0.75 amps. Current defibrillation conductor technology is required to deliver 22 amps in a single pulse. A test method was developed in which an exponential pulse of desired pulse intensity and width could be delivered across a test sample consisting of a short length of filar. The method enabled measurement of the maximum amperage supported by an individual filar prior to the point of burning out (fuse test) by techniques which are standard within the active cardiac device industry.

A test fixture was developed that would enable the testing of multiple, single filers either individually, in parallel or in sequence. A six inch segment of filar was subjected to each individual pulse test. The voltage, amperage and damage were noted for each test. Progressively increased voltage and amperage were delivered through each filar.

### Results:

The initial test run progressed in 5 volt increments up to 90 volts (the maximum capacity of our test equipment) with no burn out at a maximum of 90 volts and a current of 4.5 amps.

The second test up to 120 volts also failed to burn out the 6″ filar segment at 20 amps.

In order to burn out the filar, the tested segment length was reduced to 2″ in order to lower the impedance of the filar test segment. Maximum amperage delivered in these test runs was 18.8 Amps.

Multiple pulses at 30 volts were delivered in succession. These pulses delivered 11.6-12.8 amps. This demonstrated the burn out in this short segment under these conditions at 12.8 amps.

### CONCLUSIONS

Initial standard conductor calculations indicated a much lower current capacity of 0.75 amps than demonstrated in actual testing.

This testing demonstrated that filars fabricated as glass/silica core fibers with thin metal coatings can carry more current than standard conductors of the same cross sectional area of metal. This is one aspect of the unique conductor capabilities of the filars.

The ability of filars to carry enough current to build into a defibrillation lead is confirmed.

To deliver 22 amps of current as required for defibrillation, a minimum of two filars would be required based on calculations. This translates into a 2-3 French diameter defibrillation lead. This small size can be used as-is or additional features or structures could be added, including additional filars to increase safety margin, or details to improve handling characteristics of the defibrillation lead.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

1. An apparatus, comprising:
an electrical pulsing device configured and arranged to generate cardiac-directed electrical signals, and
a fiber optic lead configured and arranged with a bend radius that is sufficiently small to allow the fiber optic lead to extend through a coronary vein of a patient, the fiber optic lead including:
a drawn fiber core that is an insulator comprising silica or glass,
a coaxial conductive metal layer directly on the core and configured and arranged to carry the electrical signals generated by the electrical pulsing device toward a distal end of the conductor,
an electrode at the distal end of the lead, the electrode electrically connected to the coaxial conductive metal layer,
a biocompatible coating; and
an anchoring system for stabilizing the lead against unwanted migration;
wherein the fiber optic lead is sufficiently flexible to bend to a radius of about 8 to 10 times the fiber core diameter without damage.

2. The apparatus defined in claim 1, wherein the outer diameter of the fiber optic lead at the metal buffer cladding is no greater than about 750 microns.

3. The apparatus defined in claim 1, wherein the fiber core has a diameter no greater than about 450 microns.

4. The apparatus defined in claim 1, with an outer diameter no greater than about 300 microns.

5. The apparatus defined in claim 1, wherein the drawn fiber core includes a cladding comprised of glass or silica.

6. (canceled)

7. (canceled)

8. The apparatus defined in claim 1, wherein the coaxial conductive metal layer hermetically seals the fiber core.

9. (canceled)

10. (canceled)

11. The apparatus defined in claim 1, wherein the coaxial conductive metal layer is selected from the group consisting of aluminum, silver, gold, copper or platinum.

12. The apparatus defined in claim 11, wherein the coaxial conductive metal layer is between 200 nm thick and 40 microns thick.

13. The apparatus defined in claim 1, further including at least one additional fiber optic lead, each of said fiber optic leads as set forth in claim 1, and secured together in a bundle.

14. (canceled)

15. The apparatus defined in claim 1, wherein the pulsing device is configured and arranged to deliver the electrical signals at about 30 to 35 joules over a period of no more than about 25 msec.

16. The apparatus defined in claim 1, wherein the electrical pulsing device is a defibrillator.

17. The apparatus defined in claim 16, wherein the defibrillator is configured to deliver an electrical pulse of about 30 to 35 joules over a period of no more than about 25 msec.

18. The apparatus defined in claim 1, further including at least one additional fiber optic lead, each of said fiber optic leads as set forth in claim 1, and secured together in a bundle, and wherein the electrical signals are conveyed using the respective coaxial conductive metal layers configured and arranged as a single multiple filar conductor.

19. (canceled)

20. The apparatus defined in claim 1, wherein the electrical signals are cardiac therapy pulsing signals.

21. The apparatus of claim 1, further comprising a first outer polymer coating, a second coaxial conductive metal layer on the first outer polymer coating, and a second outer polymer coating on the second coaxial conductive metal layer.

22. The apparatus of claim 21 wherein the second coaxial conductive metal layer is of a different thickness than the first coaxial conductive metal layer.

23. The apparatus of claim 1, wherein the fiber core is an optical capable fiber, and further comprising an additional lower index silica or glass cladding.

24. The apparatus of claim 1, wherein the coaxial conductive metal layer is patterned to produce multiple discrete conductive paths on the same metal layer.

25. The apparatus of claim 24, wherein the coaxial conductive metal layer comprises a first and a second electrically conductive path.

26. The apparatus of claim 25, further comprising a second electrode, wherein the first electrode is electrically connected to the first electrically conductive path and the second electrode is electrically connected to the second electrically conductive path.
